(19) United States
(12) Patent Application Publication
(10) Pub. No.: US 2026/0000645 A1
(43) Pub. Date: Jan. 1, 2026

(54) USE OF EBSELEN OR ONE OF THE DERIVATIVES THEREOF TO TREAT MITOCHONDRIAL PATHOLOGIES OR DYSFUNCTIONS

(71) Applicants: ASSOCIATION FRANCAISE CONTRE LES MYOPATHIES, PARIS (FR); CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE D'ANGERS, ANGERS (FR); UNIVERSITE DE BORDEAUX, BORDEAUX (FR); UNIVERSITE PARIS-SACLAY, GIF-SUR-YVETTE (FR); UNIVERSITE COTE D'AZUR, NICE CEDEX 2 (FR); CENTRE HOSPITALIER UNIVERSITAIRE D'ANGERS, ANGERS (FR)

(72) Inventors: Déborah TRIBOUILLARD-TANVIER, CABANAC-ET-VILLAGRAINS (FR); Stéphane AZOULAY, NICE (FR); Vincent PROCACCIO, AVRILLE (FR); Carole SELLEM, PALAISEAU (FR)

(21) Appl. No.: 18/881,145

(22) PCT Filed: Jul. 5, 2023
(86) PCT No.: PCT/EP2023/068532

§ 371 Date: Jan. 3, 2025

(30) Foreign Application Priority Data
Jul. 5, 2022 (FR) FR2206822

(51) Int. Cl. A61K 31/41; A61K 9/00; C12N 3/00

(52) U.S. Cl. CPC A61K 31/41; A61K 9/0053; C12N 3/00

(57) ABSTRACT

The invention relates to the use of ebselen or one of the derivatives thereof for treating pathologies or mitochondrial malfunctions in particular associated with complex I deficiencies.

### TECHNICAL FIELD

The present invention aims to provide new pharmacological tools for treating mitochondrial illnesses or malfunctions, in particular those associated with mitochondrial complex I deficiencies.

### PRIOR ART

Mitochondrial illnesses are frequent pathologies of metabolism characterized by a high clinical and genetic heterogeneity that is manifested by the fact that mitochondria do not succeed in producing sufficient energy to allow the organism to function correctly. Mitochondrial illnesses can be present at birth but can also appear at any age. On the genetic level, mitochondria have the particularity of having their own DNA (or mitochondrial DNA) but the majority of mitochondrial proteins depend on the nuclear genome. Thereby, genetic anomalies carried by the mitochondrial genome as well as the nuclear genome are responsible for mitochondrial illnesses, reflecting the very high clinical and genetic heterogeneity. It is estimated that one person in 4300 are affected by a mitochondrial illness (Gorman et al., 2015, Ann Neurol. 77 (5): 753-9).

Mitochondrial illnesses can affect nearly every part of the body, in particular cells of the brain, the nerves, the muscles, the kidneys, the heart, the liver, the eyes, the ears or the pancreas. Mitochondrial illness symptoms depend on the organ involved, preferably affecting tissues that are high energy consumers like the muscles, the brain and the heart. The symptoms of patients can be moderate to severe, concerning one or a plurality of organs.

The symptoms of mitochondrial illnesses may include:
poor growth
muscular weakness, muscular pain, poor muscle tone, intolerance to effort
disorders of vision and/or hearing
learning difficulties, retarded development, mental retardation
autism or characteristics of autism
cardiac malfunction, cardiac arrhythmia or anomalies of cardiac conduction
illness of the liver or the kidneys
gastro-intestinal disorders, difficulties in swallowing, diarrheas or constipation, unexplained vomiting, cramps, gastro-intestinal reflux
diabetes
enhanced risk of infections
neurological disorders, epileptic seizures, migraines, cerebral vascular accidents (strokes)
disorders of mobility
thyroid and/or suprarenal gland malfunction
respiratory disorders
lactic acidosis, i.e. an accumulation of lactic acid in the blood or the urine
dementia.

Mitochondrial malfunction may also occur when mitochondria do not function properly, possibly due to another illness or another ailment. Numerous pathologies can lead to secondary mitochondrial malfunction, in particular Alzheimer or Parkinson's diseases, muscular dystrophies, Lou Gehrig's disease, diabetes and cancer. Persons suffering from a secondary mitochondrial malfunction do not suffer from a primary mitochondrial illnesses of genetic origin but have similar symptoms. Furthermore, certain drugs can target or damage mitochondria.

Mitochondrial complex I deficiency is the most common defect observed in more than 30% of mitochondrial illnesses. Amongst same, the most frequent two clinical phenotypes related to complex I deficiency are the Leigh′ syndrome that is often fatal or more moderate phenotypes such as Leber Hereditary Optic Neuropathy (LHOL). MELAS syndrome has also been considered as a common mitochondrial illness due to mutations of the mitochondrial genome and associated with a severe reduction in mitochondrial complex I activity.

Complex I is composed of at least 44 sub-units, of which seven, i.e. ND1 to 6, and ND4L are coded by mitochondrial genes, while others are coded by nuclear genes. Consequently, the clinical and molecular characteristics associated with hereditary complex I deficiency are very variable. Amongst the sub-units of complex I, it has been shown that mutations targeting the gene NDUFV1 are responsible for severe neurological phenotypes (Schuelke et al., 1999, Nat Genet. 21(3): 260-61). Mutations affecting the sub-unit NDUFS8 have been associated with Leigh's syndrome (Procaccio et al., 2004, Neurology 62:1899) and mutations targeting the sub-unit ND3 coded by mitochondrial DNA have been reported in Leigh's syndrome and in LHOL (Sarzi et al., 2007 American Journal of Medical Genetics 143: 33-41; Wang et al., 2009, Neurogenetics. 10:337-345). Furthermore, mutations affecting the sub-unit ND6 coded by mitochondrial DNA have been reported in LHOL (Johns et al., 1992, Biochem Biophys Res Commun. 187(3):1551-7).

Furthermore, complex I deficiency has been identified in secondary mitochondrial malfunction associated with neurodegenerative illnesses related to age such as Parkinson's disease.

Even if most mitochondrial illnesses have a genetic origin, genetic therapy seems difficult to implement due to the genetic and clinical diversity and the complexity of these illnesses.

The goal of current treatments is to ease the symptoms and to slow the progression of the illness or the malfunction with e.g. the following recommendations:
use of vitamin therapy
conservation of energy
slowing of activities
maintaining the environment at room temperature
protection against exposure to intercurrent illnesses (infections, stress)
maintaining a nutrition and suitable hydration.

It should be noted that the documents WO2020/254632 and WO2022/018297 have recently reported the treatment of these pathologies or deficiencies using disulfirame and alverine, respectively.

However, it is still necessary to find new therapeutic and pharmacological approaches for treating these types of malfunctions or illnesses based on causal and non-symptomatic treatment.

The document WO2014/150688 describes the treatment of cancer using molecules such as ebselen, able to inhibit enzymes from the mitochondrial chain (1-C), in particular the enzyme MTHFD2.

JIA ZHI-QIANG et al. (Neurosc. Letters, 2018, vol. 678, pp 110-17) reporting the beneficial effect of ebselen in treating an acute lesion of the spinal cord with a neuroprotective effect and an improvement of mitochondrial function.

The document ARAKAWA MOTOKI et al. (Cerebellum, 2007, 6(4), pp 308-14) relates to neurodegenerative illnesses such as Alzheimer or Parkinson's disease, described as associated with mitochondrial alterations. This document focuses on the positive effect of NAC (N-acetylcysteine) against the toxicity induced by 4-hydroxynonenal (HNE) on the neurons and mentions a neuroprotective effect of ebselen and the possible use thereof in combination with NAC.

The document AZAD GAJENDRA et al. (Molecular Biology Reports, 2014, 41(8), pp 4865-79) is a review dedicated to ebselen, described as a promising antioxidant drug. Table 2 lists all of the pathologies that can be improved by the administration of ebselen.

The document CAPPER MICHAEL et al. (Nature Communications, 2018, 9(1)) deals with the effects of ebselen on the enzyme superoxide dismutase 1 (SOD1), the mutations of which can lead to pathologies such as amyotrophic lateral sclerosis (ALS).

### DETAILED DESCRIPTION OF THE INVENTION

The inventors have shown that ebselen, a drug currently used in particular in clinical trials for the treatment of hearing disorders, such as Ménière's disease, but also its derivatives, such as ethasalen, were active in the context of the treatment of illnesses associated with a mitochondrial malfunction, in particular illnesses of the mitochondrial respiration chain, advantageously associated with a complex I deficiency. Thereby, this work opens the way for the use of a new family of compounds in this context.

### Definitions

The definitions below give the general meaning used in the framework of the invention and must be taken into account unless another definition is explicitly indicated.

The terms “about”, “roughly”, “on the order of” and “approximately” used in this document to designate a measurable value such as a quantity, a time and others should be understood as encompassing variations of ±20% or ±10%, preferably ±5%, more preferably ±1%, and again more advantageously ±0.1% with respect to the specified value.

Intervals/ranges: throughout this disclosure, miscellaneous aspects of the invention may be presented in the form of an interval of values (range format). It must be understood that the description of values in the form of an interval is only done for convenience and brevity and should not be interpreted as limiting the scope of the invention. Consequentially, the description of a range should be considered as having specifically disclosed all possible sub-ranges as well as individual numerical values within this range. For example, the description of a range such as “from 1 to 6” should be considered as having specifically disclosed the sub-ranges such as 1 to 3, from 1 to 4, from 1 to 5, from 2 to 4, from 2 to 6, from 3 to 6, etc. as well as individual numbers within this range, e.g. 1, 2, 2,7, 3, 4, 5, 5.3 and 6. This applies irrespective of the extent of the range.

“Isolated” means extracted or withdrawn from its environment or natural state. For example, an isolated nucleic acid or peptide is a nucleic acid or peptide that has been extracted from the natural environment in which same is usually found, whether e.g. in a plant or a living animal. A nucleic acid or peptide that e.g. is naturally present in a living animal is not an isolated nucleic acid or peptide within the meaning of the invention, while the same nucleic acid or peptide partially or completely separated from other components present in the natural environment thereof is itself “isolated” within the meaning of the invention. An isolated nucleic acid or peptide can exist in a substantially purified form, or can exist in a non-native environment, e.g. a host cell.

The term “abnormal” when used in the context of organisms, tissues, cells or components of same, designates organisms, tissues, cells or components of same that differ by at least one observable or detectable characteristic (e.g. age, treatment, time of day, etc.) of organisms, tissues, cells or components of same that present the respective “normal” (expected) characteristic. The characteristics that are normal or expected for one type of cell or tissue may be abnormal for another type of cell or tissue.

The terms “patient”, “subject”, “individual” and others are used interchangeably in this document and designate any animal or cell of same, in vitro or in situ, able to be subjected to the methods described in this document. In certain non-limiting embodiments, the patient, the subject or the individual is an animal, preferably a mammal, more advantageously a human, whether masculine or feminine. Said animal could also be a mouse, a rat, a pig, a dog or a non-human primate (NHP), such as a macaque monkey.

Within the meaning of the invention, an “illness” or a “pathology” is the state of health of an animal in which the homeostasis thereof is negatively affected and which continues to deteriorate if the illness is not treated. On the other hand, within the meaning of the invention a “disorder” or a “malfunction” is the state of health in which the animal is able to maintain the homeostasis thereof but in which the state of health of the animal is less favorable that it would be in the absence of said disorder. In the absence of treatment, a disorder does not necessarily lead to the deterioration of the state of health of the animal over time.

An illness or a “disorder” is “attenuated” (“reduced”) or “improved” if the seriousness of the symptom of the illness or the disorder, the frequency in which said symptom is felt by the subject, or both, are reduced. Said definition also includes the disappearance of the progression of the illness, i.e. the stopping of the progression of the illness or the disorder. An illness or a “disorder” is “cured” (“recovered”) if the seriousness of the symptom of the illness or the disorder, the frequency in which said symptom is felt by the patient or both are eliminated.

In the context of the invention, a “therapeutic” treatment is a treatment administered to a subject that has symptoms (signs) of a pathology with the goal of reducing or eliminating said symptoms. In the framework of the invention, the “treatment of an illness or a disorder” signifies the reduction of the frequency or the seriousness of at least one sign or symptom of an illness or a disorder felt by the subject. A treatment is said to be prophylactic when administered to prevent the development, the propagation or the aggravation of an illness, in particular if the subject does not present or does not yet present symptoms of the illness and/or for which the illness has not been diagnosed.

Such as used here, “treating an illness or a disorder” signifies reducing the frequency or the seriousness of at least one sign or symptom of an illness or a disorder felt by the subject. Illness or disorder are used interchangeably in the context of treatment according to the invention.

Within the meaning of the invention, an “efficacious quantity” or “an effective quantity” of a compound is the quantity of compound that is sufficient to provide the subject to which the compound is administered, with a beneficial effect. The expression “therapeutically effective quantity” refers to a quantity which is sufficient or effective for preventing or treating (in other terms retarding or preventing the development, preventing the progression, inhibiting, reducing, decreasing or reversing) an illness or a disorder, including the easing of symptoms of said illness or said disorder.

The present invention relates to the use of a compound having a moiety of formula:

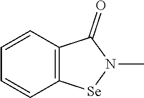

or a compound of formula:

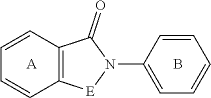

or of formula

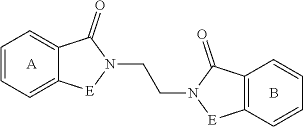

in which
E is O, S, SO (S═O), SO₂ (O═S═O), Se or SeO (Se═O)
each of the phenyl rings A and B being possibly substituted by one or a plurality of substituent,
in which each substituent is chosen independently from amongst:
   a halogen, which is preferably chosen from amongst F, Cl and Br
   an alcohol
   an amine
   a nitro
   a C1-C4 alkyl such as a C1-C2 alkyl or a C1 alkyl, possibly substituted by one or a plurality of halogen atoms, of which each is preferably chosen from amongst F, Cl and Br; and
   a C1-C4 alkoxy, such as a C1-C2 alkoxy or a C1 alkoxy, possibly substituted by one or a plurality of halogen atoms, of which each is preferably chosen from amongst F, Cl and Br
or a pharmaceutical composition containing said halogen, for the treatment of an illness associated with a mitochondrial malfunction, or for the preparation of a drug intended for the treatment of an illness associated with a mitochondrial malfunction.

In other terms, the present invention relates to a compound having a moiety of formula:

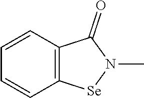

or a compound of formula:

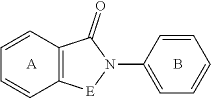

or of formula

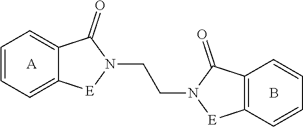

in which
E is O, S, SO (S═O), SO₂ (O═S═O), Se or SeO (Se═O)
each of the phenyl rings A and B being possibly substituted by one or a plurality of substituent,
in which each substituent is chosen independently from amongst:
   a halogen, which is preferably chosen from amongst F, Cl and Br
   an alcohol
   an amine
   a nitro
   a C1-C4 alkyl such as a C1-C2 alkyl or a C1 alkyl, possibly substituted by one or a plurality of halogen atoms, of which each is preferably chosen from amongst F, Cl and Br; and
   a C1-C4 alkoxy, such as a C1-C2 alkoxy or a C1 alkoxy, possibly substituted by one or a plurality of halogen atoms, of which each is preferably chosen from amongst F, Cl and Br
or a pharmaceutical composition containing said halogen, for use in the treatment of an illness associated with a mitochondrial malfunction.

According to another aspect, the invention relates to a method of treatment of an illness associated with a mitochondrial malfunction comprising the administration to a subject of a compound having a moiety of formula:

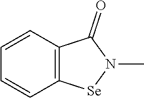

or a compound of formula:

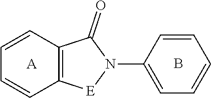

or of formula

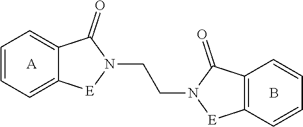

in which
E is O, S, SO (S═O), SO₂ (O═S═O), Se or SeO (Se═O)
each of the phenyl rings A and B being possibly substituted by one or a plurality of substituent,
in which each substituent is chosen independently from amongst:
   a halogen, which is preferably chosen from amongst F, Cl and Br
   an alcohol
   an amine
   a nitro
   a C1-C4 alkyl such as a C1-C2 alkyl or a C1 alkyl, possibly substituted by one or a plurality of halogen atoms, of which each is preferably chosen from amongst F, Cl and Br; and
   a C1-C4 alkoxy, such as a C1-C2 alkoxy or a C1 alkoxy, possibly substituted by one or a plurality of halogen atoms, of which each is preferably chosen from amongst F, Cl and Br
or a pharmaceutical composition comprising said halogen, for use in the treatment of an illness associated with a mitochondrial malfunction.

A compound implemented in the framework of the invention can have an activity of inhibiting inositol monophosphatase (IMPase), said activity able to be tested as described by Singh et al. (Nat Commun. 2013; 4:1332. doi: 10.1038/ncomms2320). According to a particular embodiment, such a compound is not a valproic acid or carbamazepine.

A compound implemented in the framework of the invention can have an anti-oxidant activity, said activity able to be tested as described by Noguchi et al. (Biochem Pharmacol 1992; 44: 39-44) and Nakamura et al. (J Biol Chem., 2002; 277(4):2687-94).

According to a particular embodiment, such a compound is not ascorbic acid.

According to a particular embodiment, a compound implemented in the framework of the invention having a group of formula:

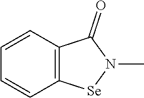

is ethaselen or one of its derivatives or analogs, as defined below.

Ethaselen or BBSKE (CAS No.: 217798-39-5) has the following formula:

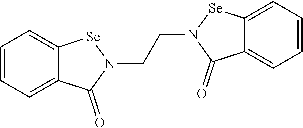 [SMILES: O=c1c2ccccc2[se]n1CCn1[se]c2ccccc2c1=O]

A derivative or analog of ethaselen is e.g. the chlorinated derivative MAD423 of formula:

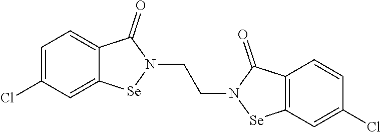 [SMILES: O=c1c2ccc(Cl)cc2[se]n1CCn1[se]c2cc(Cl)ccc2c1=O]

Thereby, a compound of interest in the framework of the invention has the following formula:

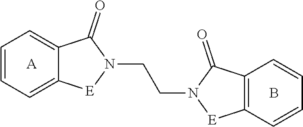

in which
E is O, S, SO, SO₂, Se or SeO
each of the phenyl rings A and B being possibly substituted by one or a plurality of substituent,
in which each substituent is chosen independently from amongst:
   a halogen, which is preferably chosen from amongst F, Cl and Br
   an alcohol
   an amine
   a nitro
   a C1-C4 alkyl such as a C1-C2 alkyl or a C1 alkyl, possibly substituted by one or a plurality of halogen atoms, of which each is preferably chosen from amongst F, Cl and Br; and
   a C1-C4 alkoxy, such as a C1-C2 alkoxy or a C1 alkoxy, possibly substituted by one or a plurality of halogen atoms, of which each is preferably chosen from amongst F, Cl and Br.

According to another embodiment, a compound implemented in the framework of the invention is as described in the document WO2012/107735, i.e. a compound of formula I, or a pharmaceutically acceptable salt of same, in which the formula I is:

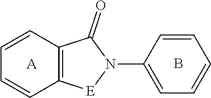

in which
E est O, S, SO, SO₂, Se or SeO
each of the phenyl rings A and B being possibly substituted by one or a plurality of substituent,
in which each substituent is chosen independently from amongst:
   a halogen, which is preferably chosen from amongst F, Cl and Br
   an alcohol
   an amine
   a nitro
   a C1-C4 alkyl such as a C1-C2 alkyl or a C1 alkyl, possibly substituted by one or a plurality of halogen atoms, of which each is preferably chosen from amongst F, Cl and Br; and
   a C1-C4 alkoxy, such as a C1-C2 alkoxy or a C1 alkoxy, possibly substituted by one or a plurality of halogen atoms, of which each is preferably chosen from amongst F, Cl and Br.

According to a preferred embodiment, such a compound is:
2-phenyl-1, 2-benzisoselenazol-3(2H)-one or ebselen of formula:

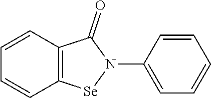 [SMILES: O=c1c2ccccc2[se]n1-c1ccccc1]

2-phenyl-1, 2-benzisothiazol-3(2H)-one or ebselen sulfur (2-phenyl-2,3-dihydro-1,2-benzothiazol-3-one) of formula:

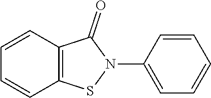 [SMILES: O=c1c2ccccc2sn1-c1ccccc1]

1-oxide-2-phenyl-1, 2-benzisoselenazol-3(2H)-one or ebselen oxide (ebselen oxide) or ebselen selenoxide of formula:

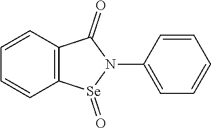 [SMILES: O=C1c2ccccc2[Se](=O)N1c1ccccc1]

ebselen —OH para 1 of formula:

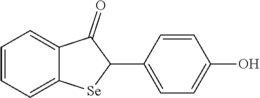 [SMILES: O=C1c2ccccc2[Se]C1c1ccc(O)cc1]

ebselen —OH ortho of formula:

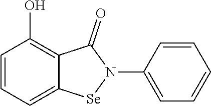 [SMILES: O=c1c2c(O)cccc2[se]n1-c1ccccc1]

ebselen —OH para 2 of formula:

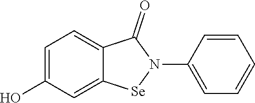 [SMILES: O=c1c2ccc(O)cc2[se]n1-c1ccccc1]

MAD281 (6-methoxy-2-phenylbenzo[d][1,2]selenazol-3(2H)-one) of formula:

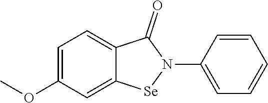 [SMILES: COc1ccc2c(=O)n(-c3ccccc3)[se]c2c1]

MAD309 (6-hydroxy-2-phenylbenzo[d][1,2]selenazol-3(2H)-one 1-oxide) of formula:

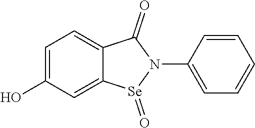 [SMILES: O=C1c2ccc(O)cc2[Se](=O)N1c1ccccc1]

MAD331 (6-chloro-2-phenylbenzo[d][1,2]selenazol-3(2H)-one) of formula:

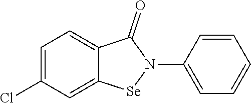 [SMILES: O=c1c2ccc(Cl)cc2[se]n1-c1ccccc1]

MAD332 (6-fluoro-2-phenylbenzo[d][1,2]selenazol-3(2H)-one) of formula:

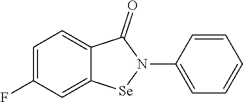 [SMILES: O=c1c2ccc(F)cc2[se]n1-c1ccccc1]

MAD349 (2-(2,6-dichlorophenylbenzo[d][1,2]selenazol-3(2H)-one) of formula:

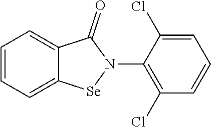 [SMILES: O=c1c2ccccc2[se]n1-c1c(Cl)cccc1Cl]

MAD380 (2-(3-hydroxyphenylbenzo[d][1,2]selenazol-3(2H)-one) of formula:

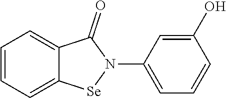 [SMILES: O=c1c2ccccc2[se]n1-c1cccc(O)c1]

MAD383 (6-chloro-2-(3-trifluoromethyl)phenyl)benzo[d][1,2]selenazol-3(2H)-one) of formula:

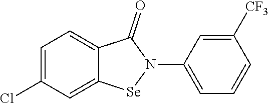 [SMILES: O=c1c2ccc(Cl)cc2[se]n1-c1cccc(C(F)(F)F)c1]

MAD384 (2-(3-nitrophenyl)benzo[d][1,2]selenazol-3(2H)-one) of formula:

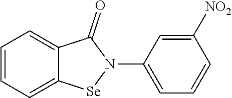 [SMILES: O=c1c2ccccc2[se]n1-c1cccc([N+](=O)[O-])c1]

MAD385 (2-(3-difluoromethoxy)phenyl)benzo[d][1,2]selenazol-3(2H)-one) of formula:

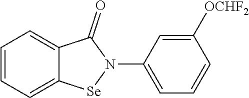 [SMILES: O=c1c2ccccc2[se]n1-c1cccc(OC(F)F)c1]

MAD413 (6-chloro-2-phenylbenzo[d][1,2]selenazol-3(2H)-one 1-oxide) of formula:

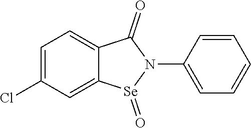 [SMILES: O=C1c2ccc(Cl)cc2[Se](=O)N1c1ccccc1]

MAD463 (6-fluoro-2-phenylbenzo[d][1,2]selenazol-3(2H)-one 1-oxide) of formula:

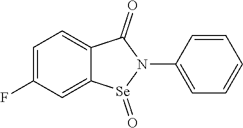 [SMILES: O=C1c2ccc(F)cc2[Se](=O)N1c1ccccc1]

Preferred compounds according to the invention are ebselen and ebselen oxide, advantageously ebselen, and their derivatives or analogs such as defined below.

Ebselen (2-phenyl-1,2-benzoselenazol-3(2H)-one; CAS number: 60940-34-3), also called PZ 51, DR3305 and SPI-1005, is a synthetic molecule able to mimic glutathion peroxidase. A therapeutic activity of this molecule has been described related to ischemic lesions, strokes, loss of hearing and bipolar disorders. It could also be effective for treating Clostridioides difficile infections and presents a fungicide activity with respect to Aspergillus fumigatus.

In practice, this molecule has been the subject of phase III clinical trials for the treatment of Ménière's disease, a hearing disorder. Ebselen is administered orally in different doses from 200 mg to 600 mg (in the form of gel-caps of 200 mg) per day for at least 21 days.

Also targeted by the present invention are derivatives or analogs of these compounds, in particular of ebselen, having the same biological activity, i.e. that reported in the examples, in particular the restoration of respiratory growth, of cell respiration or the level of mitochondrial ATP, whether in yeast models, in particular Saccharomyces cerevisiae, of filamentary molds, in particular Podospora anserina, as on human cells, in particular fibroblasts or myoblasts of patients suffering from pathologies.

The term “derivatives” (or “analogs”) encompasses derivatives and metabolites as well as pharmaceutically acceptable salts. A derivative is a compound coming from another (the precursor with a typically similar chemical structure) after transformation of the latter. The derivative can differ by one or a plurality of atoms or functional groups. A metabolite is a stable intermediate compound or a compound resulting from the biochemical transformation of an initial molecule by metabolism.

By “pharmaceutically acceptable salts”, is meant addition salts of a compound that can be obtained by reaction of said compound with a mineral or organic acid according to a method known per se. Amongst the acids traditionally used for this, one can mention hydrochloric, hydrobromic, sulfuric, phosphoric, 4-toluene sulfonic, methane sulfonic, cyclohexyl sulfamic, oxalic, succinic, formic, fumaric, maleic, citric, aspartic, cinnamic, lactic, glutamic, acids and N-acetyl-aspartic, N-acetyl-glutamic, ascorbic, malic, benzoic, nicotinic and acetic acids. According to a preferred embodiment, the compound is not in the form of a salt.

Said compounds, in particular ebselen, may be modified to increase the stability thereof, the bioavailability thereof and/or the ability thereof to reach targeted tissues, in particular the mitochondria.

In a manner known to a person skilled in the art, said compounds, in particular ebselen, may be present in the composition in a bare form (free) or contained in delivery systems that increase the stability, the targeting and/or the bioavailability, such as liposomes, or incorporated in supports such as hydrogels, cyclodextrins, biodegradable nanocapsules, bioadhesive microspheres, vectors or in combination with a cationic peptide.

The present invention also relates to pharmaceutical compositions containing as an active ingredient, at least one compound such as defined above, as well as the use of said compound or of said composition as a drug or a medicinal product.

Thereby, the present invention targets pharmaceutical compositions comprising a compound according to the invention for the targeted use. Advantageously, said compositions comprise a therapeutically effective quantity of said compound and a pharmaceutically acceptable support. In a particular embodiment, the term “pharmaceutically acceptable” means approved by a regulatory body from the federal or State government or registered in an American or European pharmacopoeia generally recognized for a use in animals and humans. The term “support” designates a diluent, an adjuvant, an excipient or a vehicle with which the therapeutic product is administered. Said pharmaceutical supports can be sterile liquids, such as water and oils, including same of petroleum origin, animal, vegetable or synthetic, such as peanut oil, soy oil, sesame oil or others. Saline solutions and aqueous solutions of dextrose and glycerol may also be used as liquid supports in particular for injectable solutions. Appropriate pharmaceutical excipients comprise starch, glucose, lactose, saccharose, sodium stearate, glycerol monostearate, talc, sodium chloride, skimmed powdered milk, glycerol, propyleneglycol, water, ethanol and others.

The composition, if need be, may also contain minor quantities of wetting agents or emulsifiers or pH buffer agents. Said compositions may take the form of solutions, suspensions, emulsions, extended release formulations and others. Examples of appropriate pharmaceutical supports are described in “Remington's Pharmaceutical Sciences” by E. W. Martin. Said compositions contain a therapeutically effective quantity of the therapeutic agent, preferably in purified form, as well as an appropriate quantity of support so as to provide a form for the adequate administration to the subject.

In a preferred embodiment, the composition is formulated in compliance with routine procedures such as an appropriate pharmaceutical composition, e.g. by oral administration to human beings. Typically, the compositions for oral administration are in the form of tablets, possibly divisible tablets or effervescent tablets, also containing excipients adapted to solid dosage form and to administration to man. As an example, ebselen can be in the form of a powder packaged in tablets able to contain 200 mg of the active ingredient. Said tablets may be crushed and mixed with liquids.

Alternatively, the composition may be in liquid form, advantageously an aqueous composition. Any other appropriate solvent may be used.

The quantity of therapeutic agent of the invention, i.e. a compound such as described above, that will be effective in the treatment of an illness, may be determined by standard clinical techniques. Furthermore, in vivo and/or in vitro trials such as those described in the examples below, may possibly be used to assist in predicting the ranges of optimum dosage. The precise dose to employ in the formulation will also depend on the administration route, the weight and the seriousness of the illness and must be decided according to the judgment of a practitioner and the conditions of each patient.

According to a particular embodiment, the composition of the invention is in solid form, advantageously a tablet that could contain 200 mg of the active compound, in particular ebselen.

According to one embodiment, the composition of the invention is in liquid form and advantageously comprises from 30 μM, 20 μM or even 15 μM of the active compound, in particular ebselen. According to one embodiment, the composition of the invention is in liquid form and advantageously comprises from 1 nM or more, even 10 nM or more, 30 nM or more, even 100 nM or more with e.g. an optimum concentration of 1 μM of active compound, in particular ebselen.

An appropriate administration route must be the delivery of an therapeutically effective quantity of the therapeutic product to the target tissues, depending on the illness.

Ebselen and the derivatives thereof may be administered in an pharmaceutically acceptable form by one of the administration routes known for this type of active ingredient.

The administration routes available are the following: topical (local), enteral (systemic action, but delivered via the gastro-intestinal (GI) tract) or parenteral (systemic action but delivered by other routes than the GI tract). In the specific case of mitochondrial illnesses, the preferred administration route of the compositions disclosed here are generally enteral, which includes oral, sublingual, buccal administration, preferably oral administration.

According to other embodiments, the administration could be parenteral administration, in particular by the intramuscular route (i.e. in the muscle) or systemic (i.e. in the circularity system). In this context, the term “injection” (or “perfusion” or “infusion”) encompasses intravascular administration, in particular intravenous (IV) and intramuscular (IM). Injections are generally performed using syringes or catheters.

According to one embodiment, the composition is administered via the oral, intramuscular, intraperitoneal, sub-cutaneous, topical, local, or intravascular route, advantageously via the oral route. According to a preferred embodiment, the composition is intended for oral administration.

A composition according to the invention is preferably in solid galenic form adapted to oral administration, advantageously in the form of one or a plurality of gelcaps or tablets.

Alternatively, the container could be in the form of liquid preparation such as elixirs and suspensions containing miscellaneous masking substances for the coloring, the taste and the stabilization.

According to a preferred embodiment, the composition is intended for oral administration. Advantageously, the composition is administered per os, i.e. via the mouth.

Preferably, a composition according to the invention is administered orally, in particular in the form of gelcaps, capsules or tablets.

To produce oral galenic forms according to the invention, in particular gelcaps, the active substance may be mixed with miscellaneous conventional materials such as starch, calcium carbonate, lactose, saccharose and dicalcium phosphate to facilitate the encapsulation process. Magnesium stearate as an additive provides a useful lubricating function if needed.

Under certain circumstances, it could be advantageous to provide controlled release forms, in particular prolonged release by means of known galenic forms.

Furthermore, a composition according to the invention may be intended for the preparation of a pharmaceutical composition that may be administered by injection.

The pharmaceutical composition according to the invention may be dissolved or put in suspension in a pharmaceutically acceptable, sterile injectable liquid, such as sterile water, a sterile organic solvent, or a mixture of said two liquids for intravenous administration.

Other routes of administration may include, but are not limited to, sub-cutaneous implants, as well as oral, sublingual, transdermal, topical, intranasal or rectal administration. Biodegradable and non-biodegradable administration systems may also be used.

As already mentioned, a composition according to the invention is preferably in solid galenic form adapted to oral administration, advantageously in the form of one or a plurality of gelcaps or tablets.

Said gelcaps or tablets may be taken with a little water before or during the main meal.

According to a preferred embodiment, the composition according to the invention is administered daily, e.g. once per day, even two or a plurality of times per day. The treatment may last a plurality of weeks, a plurality of months, a plurality of years or even a whole life.

In general, the dosage of the therapeutic agent, i.e. ebselen or one of the derivatives thereof, varies depending on factors such as age, weight, height, sex, general state of health and medical antecedents of the subject. Typically, it is desirable to provide a patient with an individual dose of the therapeutic agent, that is effective without being toxic.

According to one particular embodiment of the invention, the dose of the composition, advantageously the daily dose for a human to take orally, is less than or equal to 10 mg or 9, 8, 7, 6, 5, 4, 3 mg/kg, even less than or equal to 2.5, 2, 1.5 or 1 mg/kg, even less than or equal to 0.9, 0.8, 0.7, 0.6, 0.5, 0.4, 0.3 or 0.1 mg/kg.

As already indicated, the patient is advantageously a human, in particular a new-born, a young child, a child, an adolescent or an adult, irrespective of the sex thereof. The therapeutic tool according to the invention can however be adapted and useful for the treatment of other animals, in particular the pig, the mouse, the dog or the macaque monkey.

In general, the present invention relates to the treatment of mitochondrial illnesses in general, i.e. illnesses related to or caused by a mitochondrial malfunction. In the framework of the present application, the expression “illness associated with a mitochondrial malfunction” is used in order to encompass all said situations.

In relation to the examples below demonstrating a positive effect of ebselen or of one of the derivatives thereof on a mitochondrial respiratory chain, the illnesses presenting a particular interest are illnesses of the mitochondrial respiratory chain.

A plurality of mitochondrial illnesses have been documented in the prior art:

NARP syndrome (neuropathy, ataxia and retinitis pigmentosa) induces a variable combination of retarded development, retinitis pigmentosa, dementia, epileptic fits, ataxia, proximal neurogenic muscular weakness and of sensory neuropathy. Said NARP syndrome is caused by miscellaneous mutations of the mitochondrial gene ATP6, which codes the sub-unit a of ATPase (complex V of the OXPHOS system). The mutations often display heteroplasmy (co-existence of mutated and wild type (WT) mitochondrial DNA (mtDNA) in the same cell). Depending on the type of mutation and the percentage of mutated mtDNA (degree of heteroplasmy), the clinical consequences are of variable severity. ATP6: m.8993T>C/G mutations are amongst the most frequent in NARP patients leading to severe forms of NARP syndrome. FMC1 is a nuclear gene which codes a protein required at high temperature (35-37° C.) for the assembly of the sector F1 of ATP synthase, thereby mimicking the heteroplasmy observed in patients with NARP. Indeed, when same are cultivated at a restrictive temperature (35-37° C.), the mitochondria of the mutant fmc1Δ contain much less assembled ATP synthase complex than a wild type (WT) strain, but those that are assembled are fully functional. This heterogeneity is also found in patients having reduced levels of ATP synthase due to heteroplasmic mutations of the ATP6 gene. Consequently, the mutant fmc1Δ constitutes an appropriate model for this illness, in particular equivalent to the mutant m.8993T<G (MR14, NARP) (Schon E A et al. (2001) Cell Dev Biol. 12(6):441-8).

The TAZ gene codes tafazzin, a mitochondrial transcylase that catalyzes the remodeling of immature cardiolipin into its mature composition containing a predominance of tetralinoleoyl fragments. <3980>TAZ</3980> mutations lead to Barth's syndrome, an illness related to the X chromosome, traditionally characterized by a dilated cardiomyopathy (CMD) with endocardial fibroelastosis (EFE), a proximal predominant skeletal myopathy, a failure to thrive, neutropenia and organic aciduria, in particular an excess of 3-methylglutaconic acid (Barth, P. G. et al. (1996) J Inherit Metab Dis, 19, 157-160).

The genes COX2 and SURF1 code a sub-unit and an assembly factor of mitochondrial complex IV, respectively. The mutations are associated with Leigh's syndrome, a severe, progressive neurodegenerative illness that appears in the first months and first years of life and responsible for early death. Persons affected generally have overall development retardation or a development regression, hypotonia, ataxia, distonia and ophthalmological anomalies, such as nystagmus or optical atrophy (Barrientos, A. et al. (2002) EMBO J, 21, 43-52).

MPV17 code a protein of the internal mitochondrial membrane, the function of which is unknown. MPV17 mutations lead to:
The mitochondrial-6 DNA depletion syndrome, a recessive autosomal illness characterized by the appearance in an infant of a progressive hepatic insufficiency, often leading to death in the first year of life. Those infants that survive develop progressive neurological damage, in particular ataxia, hypotonia, dystonia and a psycho-motor regression (Spinazzola, A. et al. (2006) Nat Genet, 38, 570-575).
Navajo type neuropathy: The manifestations comprise a severe anesthesia lead to ulceration of the cornea, non-painful fractures, acral mutilation, muscular weakness, absence or clearly diminished of deep tendon reflexes without intellectual deficit (Karadimas, C. L. et al. (2006) Am J Hum Genet, 79, 544-548).

Of particular interest is the treatment of an illness chosen from amongst the group composed of: MELAS syndrome, myopathy or cardiomyopathy inherited from the mother, NARP or MILS syndrome, Leigh's syndrome, Leber hereditary optic neuropathy (LHON), Barth's syndrome, the depletion of mitochondrial DNA syndrome in particular 4A (Alpers type) and 4B (MNGIE type), mitochondrial recessive ataxia syndrome, sensory ataxic neuropathy, dysarthria and ophthalmoplegia, spinocerebellar ataxia with epilepsy, progressive external ophthalmoplegia, mitochondrial DNA-6 depletion syndrome, Navajo type neuropathy, Behr's syndrome, mitochondrial DNA 14 depletion syndrome, infantile cardioencephalomyopathy due to a deficit of cytochrome c oxydase (COA6 mutations), mitochondrial type 1 complex III nuclear deficiency, GRACILE syndrome and Bjornstad's syndrome.

The treatment of illnesses associated with mitochondrial complex I deficiency or deficiencies presents a particular interest. Certain illnesses are uniquely linked to a complex I malfunction while other illnesses are associated with multiple deficits e.g. affecting a plurality of mitochondrial complexes.

In a known manner, the respiratory chain in mitochondria is involved in oxidative phosphorylation, which is an important cellular process that uses oxygen and simple sugars to create adenosine triphosphate (ATP), the main source of energy for the cell. Five protein complexes, constituting the OXPHOS system, composed of a plurality of proteins each, are involved in this process. Said complexes are called complex I, complex II, complex III, complex IV and complex V respectively. Complex I (CI or NADH dehydrogenase or NADH co-enzyme Q reductase), the first enzyme of the respiratory chain, is a very large protein complex (about 1000 kDa) composed of at least 44 sub-units including 7 coded by mitochondrial DNA (ND1 to ND6 and ND4L). According to a particular embodiment, a compound according to the invention, in particular ebselen, can be used for treating a so-called “primary” mitochondrial illness, i.e. due to an identified genetic anomaly identified in at least one sub-unit of OXPHOS complexes (in particular the complex I) related to one or a plurality of mitochondrial or nuclear DNA mutations. These pathologies are associated with neurological, cardio-muscular or ophthalmological symptoms related to tissues or organs that are the most affected in these mitochondrial illnesses, even if other organs or tissues are possibly affected.

According to a particular embodiment, a compound according to the invention, in particular ebselen, can be used to treat a so-called “secondary” mitochondrial illness. In this case, the genetic anomaly does not directly involve the OXPHOS complexes but the pathology will affect mitochondrial functions and in particular can lead to a reduction of the enzymatic activity of this process. Such an illness can also be due to genetic causes such as exposure to environmental factors or aging. This is the case in particular of Parkinson's disease or other neurodegenerative disorders related to age.

According to a preferred embodiment, the deficiency or the mitochondrial malfunction results from a genetic illness.

Genetic illnesses are, by definition, illnesses resulting from one or a plurality of genetic defects (anomalies) (or mutations) in one or a plurality of genes. Genetic defects can affect mitochondrial DNA and/or nuclear genes. Genetic defects responsible for mitochondrial illnesses can be isolated mutations leading to a change of codon.

However, the illnesses can be related to the deletion or insertion of one or a plurality of bases or codons.

According to a specific embodiment, the illness results from one or a plurality of genetic deficiencies (or mutations) in one or a plurality of genes involved in the functionality of OXPHOS complexes, in particular complex I.

A non-limiting list of such genes comprises:
complex I structural genes, in particular MTND1 (or ND), MTND2 (or ND2), MTND3 (or ND3), MTND4 (or ND4), MTND5 (or ND5), MTND6 (or ND6), MTND4L (or ND4L), NDUFA1, NDUFA2, NDUFA3, NDUFA4, NDUFA5, NDUFA6, NDUFA7, NDUFA8, NDUFA9, NDUFA10, NDUFA11, NDUFA12, NDUFA13, NDUFAB1, NDUFB1, NDUFB2, NDUFB3, NDUFB4, NDUFB5, NDUFB6, NDUFB7, NDUFB8, NDUFB9, NDUFB10, NDUFB11, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3;
complex I assembly genes, in particular NDUFAF1, NDUFAF2, NDUFAF3, NDUFAF4, NDUFAF5, NDUFAF6, NDUFAF7, NDUFAF8, NUBPL, ACAD9, TMEM70, TMEM126B, FOXRED1, ECSIT, AIF, TIMMDC1.

According to a particular embodiment, the illnesses to treat in the framework of the invention are related or due to at least one genetic defect or mutations in at least one of the following genes: MTTL1, ATP6, FMC1, TAZ, COX2, SURF1, POLG, MPV17, OPA1, COA6, ND6 and BCS1L, advantageously ATP6, TAZ, COX2, SURF1.

A plurality of mitochondrial illnesses of genetic origin, in particular in relation to deficiencies of mitochondrial complex I, have been documented in the prior art: LHON syndrome or Leber Hereditary Optic Neuropathy generally appears in young adults. The start is abrupt with a rapid lowering of vision in the center of the eye, corresponding to a decrease in central visual acuity. Most often, a peripheral visual field persists as a vision halo around a blind zone. This illness is due to communal homoplasmic mutations in genes coding for sub-units of complex I of the respiratory chain. In practice, mitochondrial DNA mutations m.11778G> A, m.3460G> A and m.14484T> C represent about 95% of LHON mutations.

Leigh's syndrome (or LS) is a serious, neurodegenerative illness. Persons affected generally have overall development retardation or a development regression, hypotonia, ataxia, distonia and ophthalmological anomalies, such as nystagmus or optical atrophy. Leigh's syndrome can also have harmful multi-systemic effects on cardiac, hepatic, gastro-intestinal and renal organs. Biochemical investigations on patients suffering from Leigh's syndrome have a tendency to display an increase of lactate and anomalies of mitochondrial oxidative phosphorylation. Leigh's syndrome can be associated with mutations in genes coding for sub-units of complex I such as the mutations NDUFV1 or the mutation MTND5 m.13513G>A.

MELAS syndrome comprising mitochondrial myopathy, encephalopathy, lactic acidosis and Stroke type episodes, is a genetically heterogeneous mitochondrial illness, with a variable clinical phenotype. This disorder is accompanied by central nervous system damage characteristics, in particular epileptic fits, hemiparesis, hemianopsia, cortical blindness and episodic vomiting. This syndrome was first of all associated with the mutation m.3243A>G of mitochondrial DNA, i.e. of the gene tRNA^{Leu (UUR)} (MTTL1), which induces an alteration of the translation of mRNA of complex I in proteins and hence a reduction in the quantity of structural proteins of complex I such as the mitochondrial sub-unit ND6. MELAS syndrome can also be associated with other mitochondrial DNA mutations such as the mutation m.3260A>G, which also affects tRNA^{Leu (UUR)}. Said mutation m.3260A>G may also lead to other clinical phenotypes, in particular a myopathy and a cardiomyopathy of maternal origin.

Thereby, the treatment of genetic illnesses that have been demonstrated to be associated with a complex I deficiency, e.g. MELAS syndrome, Leigh's syndrome and Leber Hereditary Optic Neuropathy (LHON) have a particular interest. It should be noted that said illnesses may be associated with other symptoms such as cardiac, myopathic or neurological clinical phenotypes.

More generally, a compound according to the invention, in particular ebselen, may be used to treat the mitochondrial malfunction, in particular, the mitochondrial malfunction associated with complex I deficiencies. The mitochondrial malfunction characterized by a loss of efficiency of the electron transport chain and reductions in the synthesis of high energy molecules like adenosine-5′-triphosphate (ATP) is a characteristic of aging and generally of all chronic illnesses.

Said illnesses include:
neurodegenerative illness such as Alzheimer, Parkinson's disease, Huntington's disease, amyotrophic lateral sclerosis (Lou Gherig's disease) and Friedreich's ataxia;
cardiovascular illnesses, such as atherosclerosis and other cardiac and vascular ailments;
diabetes and metabolic syndrome;
auto-immune illnesses, such as multiple sclerosis, systemic lupus erythematosus and type 1 diabetes;
neurobehavioral and psychiatric illnesses, such as autistic spectrum disorders, schizophrenia, bipolar and mood disorders;
gastro-intestinal disorders;
fatigue illnesses such as chronic fatigue syndrome and Gulf War illnesses;
muscular-skeletal illnesses such as fibromyalgia and hypertrophy/atrophy of the skeletal muscles;
muscular dystrophies,
cancer; and
chronic infections.

The treatment recommended by the present invention, based on ebselen or on one of the derivatives thereof, may also be associated with other treatments, intended to treat the same pathology or the same condition or another illness.

According to a particular embodiment, said other treatment is based on the administration of another compound of interest.

Thereby and according to this aspect, the composition according to the invention is associated with at least one other compound for the treatment of the same illness. The composition according to the invention and said compound could be administered simultaneously or separately over time to take into account the particularities thereof and in particular the bio-availabilities thereof.

According to one particular mode of application, the present invention relates to a composition, advantageously a pharmaceutical composition or a drug containing a compound such as described above and potentially other active molecules (other genetic therapy molecules, chemical moieties, peptides or proteins) for the treatment of the same illness or of a different illness, advantageously the same illness.

Preferably, the pharmaceutical composition according to the present invention and at least one compound for the treatment of the same illness or a different illness, are administered simultaneously, separately or spread out over time for treating the same illness or a different illness.

More generally, with regard to mitochondrial illnesses, another compound able to improve the mitochondrial function may be administered simultaneously or at different times. In the case of simultaneous administration, the two compounds can be associated in the same composition.

Examples of such additional compounds are natural supplements, such as L-carnitine, alpha-lipoic acid (α-lipoic acid [1,2-dithiolane-3-pentanoic acid]), coenzyme Q10 (CoQ10 or ubiquinone), riboflavin (vitamin B2) reduced in nicotinamide adenine dinucleotide (NADH), L-arginine, possibly in combination.

Examples of compounds used e.g. in the case of MELAS syndrome are nitric oxide (NO) precursors such as L-arginine and citrulline.

According to a particular embodiment and in view of the examples demonstrating a potential synergistic effect, a compound according to the invention, in particular ebselen, was combined with one of the compounds described in the documents WO2020/254632 and WO2022/018297, advantageously chosen in the group constituted by: disulfirame, sodium diethyldithocarbamate, alverine and alverine citrate.

Subjects who could benefit from the compositions according to the invention comprise all patients having an illness associated with a mitochondrial malfunction, in particular a mitochondrial malfunction associated with complex I deficiencies, diagnosed as carriers of such an illness or risking developing such an illness.

A subject to treat with a composition according to the invention may be chosen on the basis of different criteria. With regard to mitochondrial malfunction, in particular complex I deficiencies, a plurality of tests could be performed, e.g.:
on the biochemical level, O₂ consumption and/or the mitochondrial complex I activity can be measured from a biopsy from the subject, in particular muscular or cutaneous. The activity of other complexes of the respiratory chain may also be evaluated to determine if the mitochondrial malfunction is only due to complex I deficiencies:
at the genetic level: the sequencing of mitochondrial or nuclear DNA, extracted from the blood, from cells or from a biopsy sample, e.g. from the skin, will allow one or a plurality of molecular anomalies to be identified, in particular mutations or deletions/insertions in the genes in particular numerated below. Alternatively, the expression or the activity of corresponding proteins may be evaluated by any method known to a person skilled in the art (e.g. by western blot).

The goal of the invention is to provide a safe (non-toxic) treatment. Another goal is to provide an effective treatment that will allow the development of the illness to be retarded, slowed or prevented and possibly to improve the phenotype of the patient that can be monitored at the clinical level as indicated below.

With a subject, the composition according to the invention can be used:
to improve mitochondrial function, in particular, mitochondrial respiration;
to improve growth;
to improve muscular function;
to improve vision and/or hearing;
to improve respiratory function;
to improve cardiac, hepatic or renal functions;
to improve cerebral functions;
to improve digestive function; and/or
to prolong survival, more generally for improving the quality and expectancy of life.

According to one aspect, the invention relates to a method for improving mitochondrial function, in particular, complex I activity, advantageously without undesirable effects, comprising the administration to a subject that needs a therapeutic quantity of a composition such as described above.

Advantageously, said improvements are observed up to 1 month after the start of treatment or 3 months or 6 months or 9 months, more advantageously up to 1 year after the start of treatment, 2 years, 5 years, 10 years or even throughout the life of the subject.

In one embodiment, said improvements appear as a reduction of the severity and/or the frequency of symptoms and/or retarded appearance.

An improvement can be assessed on the basis of methods known in the art, e.g. in the case of MELAS:
evaluation of the quantity of lactate, in particular cerebral ventricular lactate, measured e.g. by magnetic resonance spectroscopy (MRS);
evaluation of the quality and/or the expectancy of life by clinical scales, e.g. the NMDAS (Newcastle Mitochondrial Disease Scale for Adults) score or the SF-36 (Short Form Health Survey) score;
evaluation of brain modifications e.g. using magnetic resonance imaging (MRI);
evaluation of muscular activity modifications by means of physical tests such as the six minute walking test;
evaluation of venous lactate changes and the concentration of GDF 15;
evaluation of mtDNA heteroplasmy modifications in the urine and blood.

Suitable parameters for a given case can be adapted depending on the illness. Thereby, the treatment claimed allows the clinical condition and the different parameters disclosed above to be improved with respect to an untreated subject.

The implementation of the present invention uses, unless otherwise indicated, conventional techniques of molecular biology (including recombination techniques), of microbiology, cellular biology, biochemistry and immunology, known to a person skilled in the art. These techniques are explained in detail in the literature, in particular in “Molecular Cloning: A Laboratory Manual”, fourth edition (Sambrook, 2012); “Oligonucleotide Synthesis “(Gait, 1984); “Culture of Animal Cells” (Freshney, 2010); “Methods in Enzymology”“Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting” (Babar, 2011); “Current Protocols in Immunology” (Coligan, 2002). Particularly useful techniques for particular implementation modes will be discussed in the following sections.

The disclosures of this patent, patent application and publication cited in the present application are incorporated by reference to the completeness thereof.

Without other description, it is considered that a person skilled in the art may, by using the description and the following illustrative examples, produce and use the compounds of the present invention and implement the claimed methods.

### EXAMPLES OF IMPLEMENTATION

The invention and the advantages that arise from same will be made clearer by the following examples of implementation supported by the associated figures. These, however, do not have any limited bearing.

### FIGURE CAPTIONS

FIG. 1:

A/ Effect of ebselen (EBS) on the respiratory growth of a plurality of yeast models of mitochondrial illnesses on non-fermentable solid medium, detected by the halos test

C/ Effect (detected by the halos test) of ebselen oxide on the respiratory growth of the mutant yeast strain taz1 on non-fermentable solid medium, in comparison with ebselen (B) and DMSO (D)

E/ Effect of different analogs of ebselen (EBS) and ethaselen on the respiratory growth of yeast strains taz1, fmc1, cox2 and A29G.

FIG. 2: Effect of ebselen (EBS) on mutant yeast strains taz1

A/ Respiratory growth in liquid medium

B/ Accumulation of Cox2 protein

FIG. 3: Effect of ebselen (EBS) on the mutant yeast strain sym1

A/ Respiratory growth in liquid medium

B/ Cellular respiration

C/ Accumulation of Cox2 protein

FIG. 4: Effect of EBS on heat sensitive growth of a model of carrying a mutation of mitochondrial complex I (Pa nuo-51^{A357V})

A/ Effect on growth halo

B/ Dose-dependent effect on growth

FIG. 5: Effect of EBS on the proliferation and viability of cells of a TAZ patient

A/ growth of fibroblasts of TAZ patients in presence or absence of EBS (12.15 nM)

B/ respiratory growth at 96 hours of fibroblasts of TAZ patients as a function of EBS concentration (from 150 μM to 234.9 μM), compared to the negative control DMSO.

C/ respiratory growth at 96 hours of fibroblasts of TAZ patients as a function of ethaselen concentration (from 150 μM to 26.1 μM) compared to the negative control DMSO.

D/ level of mitochondrial ATP in fibroblasts of TAZ patients incubated in presence of Antimycin A (100 μM), Rotenone (0.5 μM) or oligomycin A (3 μM) and EBS in a concentration of 12.15 nM.

E/ respiratory growth at 120 hours of myoblasts of a TAZ patient as a function EBS concentration (from 50 μM to 234.9 μM).

FIG. 6: Effect of EBS on the proliferation and viability of a HeLa cell line, in which the gene FMC1 has been deleted, in glucose 1 g/L (A) or galactose (B) medium D.O. (AU): optical density and arbitrary units

FIG. 7: Effect of EBS on the mitochondrial respiration of cells of patients deficient for the complex I (CI) of the mitochondrial respiratory chain, carrying mutations affecting the nuclear gene NDUFV1 of the complex

A/ oxygen consumption rate (OCR)

B/ basal respiration (routine)

C/ respiration related to ATP synthase (R-O)

D/ maximum respiration

FIG. 8: Synergistic effects between ebselen (EBS), disulfirame (DSF) and alverine (ALV)

A/ in a taz yeast mutant model

B/ in a mutant Pa nuo-51^{A357V}: speed of growth in presence of EBS and ALV alone or in combination, compared to negative control DMSO (VEH)

FIG. 9: Effect of taking EBS on the weight of KO mice lacking the TAZ gene (mouse model for Barth syndrome)

### EXAMPLES 1 TO 3: SACCHAROMYCES CEREVISIAE MODEL

Each of the strains of the yeast Saccharomyces cerevisiae used in these examples contain different specific mutations modeling human mutations leading to mitochondrial illnesses. At varying degrees, all these mutant yeast strains have a growth defect when cultivated on a respiratory medium such as ethanol or glycerol at 28° C. or 36° C. (depending on the strain).

### Mutated Yeast Strains:

fmc: MC6 MATa ade2-1 his3-11,15 trp1-1 leu2-3,112 ura3-I fmc1: :HIS3 [Δi ER OR] (Schwimmer, C. et al. (2005) J Biol Chem, 280, 30751-30759) for the first screening
MR14 MATa ade2-1 his3-11,15 trp1-1 leu2-3,112 ura3-1 CAN1 arg8::HIS3 ρ+ atp6-L173R or RKY20MATa ade2-1 his3-11,15 trp1-1 leu2-3,112 ura3-1 CAN1 arg8::HIS3 ρ+ atp6-L173P (Rak, M. et al. (2007) J Biol Chem, 282, 34039-34047) for the second screening
taz1Δ: this strain was produced by substituting the open reading framework of TAZ1 by that of TRP1 in the strain W303-1A (MATa ade2-1 ura3-1 his311, 15 trp1-1 leu2-3, 112 can1-100) (from Taffin de Tilques, M. et al. (2017) Dis Model Mech, 10, 439-450)
shy1-G137R: this mutant was produced in the strain CW252 containing the nuclear base of W303 and a mitochondrial gene lacking intron. This gene is a homologue of the human gene SURF1
sym1Δ: this yeast strain was produced by substituting the open reading framework of SYM1 by that of kanMX6 in the strain W303-1A (MATa ade2-1 ura3-1 his311, 15 trp1-1 leu2-3,112 can1-100). This gene is a homologue of the human gene MPV17
cox2: MATa lys2 leu-2-3,112 ura3-52 his3ΔHindIII arg8::hisG cox2-22 (Bonnefoy, N. et al. (2001) Mol Cell Biol, 21, 2359-2372.
A29G: The yeast strain used in these experiments includes tRNA-Leu A29G with a genetic base MATa, his3-11, ade2-1, leu2-3,112, ura3-1, trp1-D2, can1-100, syn-.

### Associated Mitochondrial Illnesses:

NARP syndrome NARP syndrome (neuropathy, ataxia and retinitis pigmentosa) induces a variable combination of the retarded development, of retinitis pigmentosa, of dementia, of epileptic fits, of ataxia, of proximal neurogenic muscular weakness and of sensory neuropathy. NARP is caused by miscellaneous mutations of the mitochondrial gene ATP6, that codes a sub-unit a of ATPase (complex V of the OXPHOS system). The mutations often display heteroplasmy (co-existence of mutated and wild type (WT) mitochondrial DNA (mtDNA) in the same cells). Depending on the type of mutation and the percentage of mutated mtDNA (degree of heteroplasmy), the clinical consequences are of variable severity. ATP6: m.8993T>C/G mutations are amongst the most frequent in NARP patients leading to severe forms of NARP syndrome. FMC1 is a nuclear gene which codes a protein required at high temperature (35-37° C.) for the assembly of the sector F1 of ATP synthase, thereby mimicking the heteroplasmy observed in patients with NARP. Indeed, when same are cultivated at a restrictive temperature (35-37° C.), the mitochondria of the mutant fmc1A contain much less assembled ATP synthase complex than a wild type (WT) strain, but those that are assembled are fully functional. This heterogeneity is also found in patients having reduced levels of ATP synthase due to heteroplasmic mutations of the ATP6 gene. Consequently, the mutant fmc1Δ constitutes an appropriate model for said disorders (Lefebvre-Legendre, L. et al. (2001) J Biol Chem, 276, 6789-6796). Miscellaneous NARP yeast strains, carrying homoplasmic mutations equivalent to mutations T8993G and T8993C (Rak, M. et al., J Biol Chem. 2007 282(47):34039-47), have also been tested.

### TAZ

The TAZ gene codes tafazzin, a mitochondrial transcylase that catalyzes the remodeling of immature cardiolipin into its mature composition containing a predominance of tetralinoleoyl fragments. <6920>TAZ</6920> mutations lead to Barth's syndrome, an illness related to the X chromosome, traditionally characterized by a dilated cardiomyopathy (CMD) with endocardial fibroelastosis (EFE), a proximal predominant skeletal myopathy, a failure to thrive, neutropenia and organic aciduria, in particular an excess of 3-methylglutaconic acid (Barth, P. G. et al. (1996) J Inherit Metab Dis, 19, 157-160).

### COX2 AND SURF1

The genes COX2 and SURF1 (SHY1 gene in yeast) code a sub-unit and an assembly factor of mitochondrial complex IV, respectively. The mutations are associated with Leigh's syndrome, a severe, progressive neurodegenerative illness that appears in the first months and first years of life and can lead to early death. Persons affected generally have overall development retardation or a development regression, hypotonia, ataxia, distonia and ophthalmological anomalies, such as nystagmus or optical atrophy (Barrientos, A. et al. (2002) EMBO J, 21, 43-52).

MPV17 MPV17 (SYM1 gene in yeast) codes a protein of the internal mitochondrial membrane the function of which is unknown. MPV17 mutations lead to:
The mitochondrial-6 DNA depletion syndrome, a recessive autosomal illness characterized by the appearance in an infant of a progressive hepatic insufficiency, often leading to death in the first year of life. Those infants that survive develop progressive neurological damage, in particular ataxia, hypotonia, dystonia and a psycho-motor regression (Spinazzola, A. et al. (2006) Nat Genet, 38, 570-575).
Navajo type neuropathy: The manifestations comprise a severe sensory-motor peripheral neuropathy with hepatic and cerebral damage such as leucoencephalopathy and corneal ulcerations. (Karadimas, C. L. et al. (2006) Am J Hum Genet, 79, 544-548).
MELAS syndrome: The mutation A30(29)G in yeast A29G mimics the human mutation m.3260A>G of the gene tRNALeu (UUR) responsible for MELAS syndrome.

### Example 1: Effect of Ebselen (EBS) and the Derivatives Thereof on Mutant Yeast Strains Cultivated in a Non-Fermentable (Respiratory) Environment

### Materials and Methods

As previously described (WO2020/254632), the different mutant yeast strains were spread over the solid respiratory agar-agar based media (containing glycerol or ethanol as the unique carbon source) and exposed to filters on which the compound tested, in particular ebselen (EBS) was deposited. The dishes were then incubated at the temperature indicated (that could be 28° C. or 36° C. depending on the strain used).

More precisely, 0.125 OD of mutant cells with exponential growth were spread homogeneously with sterile glass beads in a square Petri dish (12 cm×12 cm) containing a solid respiratory medium (YPA Ethanol, 1% yeast extract, 0.5% Bacto Peptone, 40 mg/L Adenine, 2% Ethanol for sym1 and taz1; or YPA Glycerol 2% for cox2, fmc1, atp6, shy1 et A29G). Small sterile filters were then placed on the agar-agar surface and growth concentration of the compounds to test were added to the filters in the quantities indicated (30 and 90 nmoles). On each dish, the DMSO (buffer) was used as negative control (upper left-hand filter). The dishes were then incubated at 28° C. (for shy1) or 36° C. (for cox2, sym1, fmc1, atp6 taz1 and A29G) for 4 to 5 days, then photographed. The improvement of the growth was evaluated after a plurality of days of incubation by the appearance of a halo of improved growth around the filter where the active ingredient was deposited.

### Results

As can be seen in Figure TA, the EBS is active on all the mutant strains of yeast tested in a dose dependent manner.

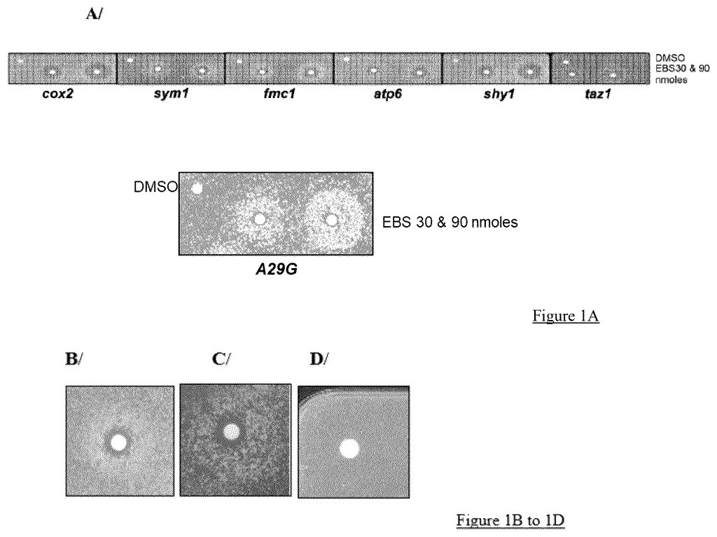

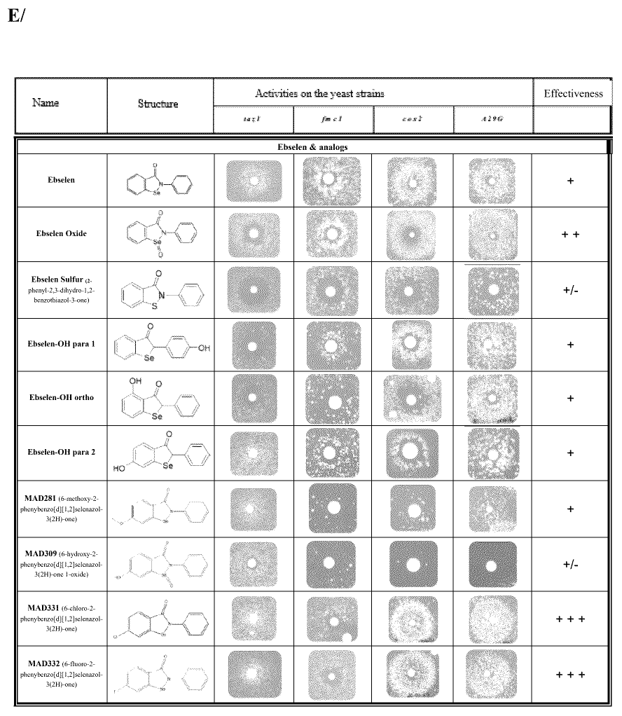

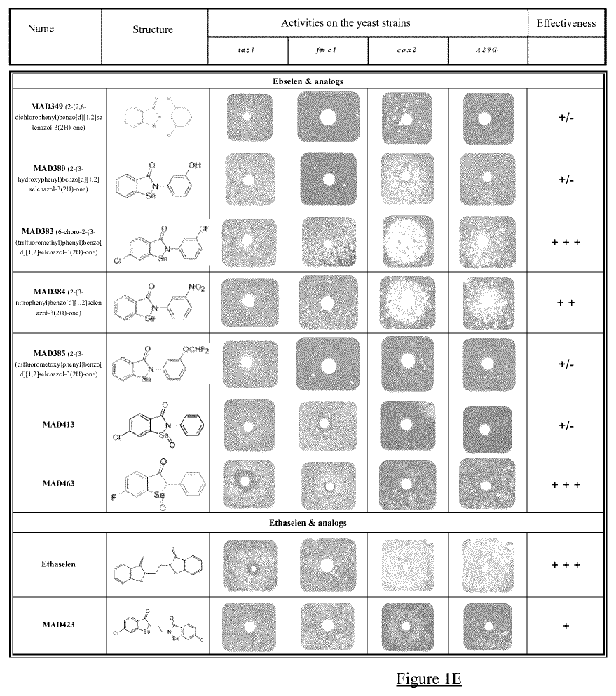

For the mutant taz1, FIG. 1C shows that ebselen oxide (100 nmoles) has an activity comparable to ebselen (100 nmoles) (FIG. 1B), while no halo was observed in the presence of DMSO (FIG. 1D).

More generally, different analogs of EBS and ethaselen were evaluated on different mutants (taz1, fmc1, cox2 and A29G). As shown in FIG. 1E, all analogs tested are active on the strains tested.

### Example 2: Effect of Ebselen (EBS) on Mutant Yeast Strains Taz1

### Materials and Methods

### Respiratory Growth in Liquid Culture:

In order to determine the optimum concentration of EBS leading to the suppression of a respiratory growth defect of the mutant yeast strain, cells undergoing exponential growth were inoculated in fresh Ethanol YPA non-fermentable media supplemented or not supplemented with increasing concentrations of EBS. The growth of cells in the liquid respiratory medium was monitored using the bioscreen system over a period of 80 h during which (OD600 nm) cell densities were sampled every 20 minutes.

### Cellular Respiration:

Respiratory intensity corresponds to the amount of oxygen consumed with respect to time and the number of cells. It reflects the mitochondrial oxidative metabolism of the cells. The consumption of oxygen was measured using the OROBOROS oxygraph system. The cells were cultivated over 7-8 generations at 28° C. for 24 to 48 h in an Ethanol YPA medium supplemented by DMSO or EBS (1 μM). 10⁷ cells were introduced into the oxygraph. The consumption of O₂ was recorded with or without CCCP (maximum rate of oxygen consumption). The rate of O₂ consumption was calculated on the basis of the linear part of the O₂ consumption.

### Accumulation of Cox2 Protein:

Total protein extracts from the mutant yeast strain were analyzed by SDS-PAGE (50 pg per well) using antibodies against the indicated proteins. The gels shown are representative of at least 3 experiments. Protein levels were quantified using the ImageJ software. The levels of Cox2 were normalized with respect to Ade13p and expressed with respect to the wild type (WT) strain.

### Results

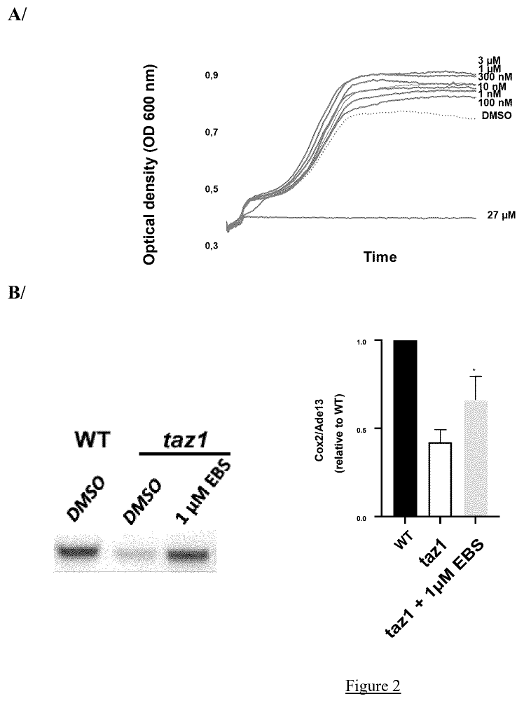

As shown in FIG. 2A, EBS is toxic as of 27 μM and active between 1 nM and 3 μM with an optimum concentration of 1 μM on the mutant strain taz1.

Furthermore and as illustrated in FIG. 2B, 1 μM of EBS increases the accumulation of the protein Cox2, a sub-unit of complex IV of the mitochondrial respiratory chain in the mutant strain taz1.

### Example 3: Effect of Ebselen (EBS) on the Mutant Yeast Strain Sym1

The experiments described in example 2 were implemented on the mutant yeast strain sym1.

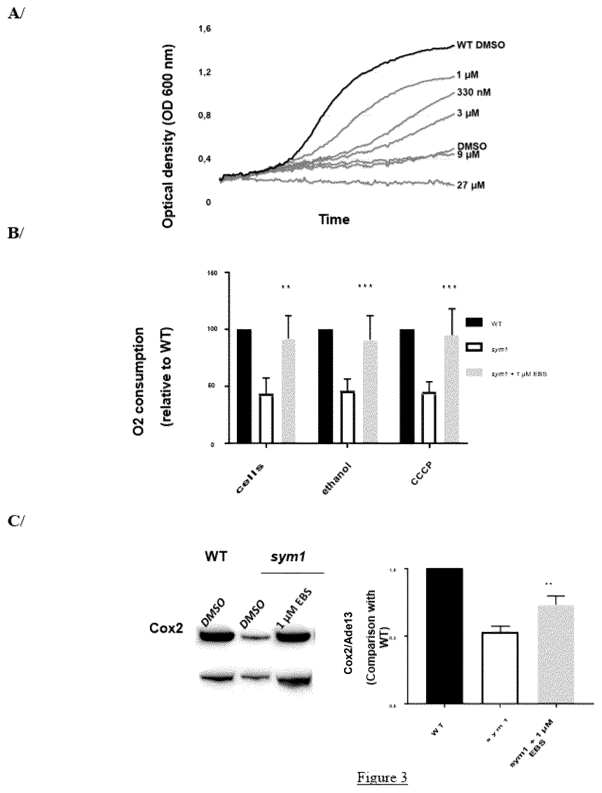

The results are shown in FIG. 3.

The data are the averages ±SEM of at least three independent experiments. The significance of the variations between samples and the controls were estimated using the multi-factorial Anova: Tukey test.

As for the mutant strain taz1, at the optimum concentration of EBS (1 μM), the cell respiration and the level of Cox2 were increased compared to non-treated cells.

### Example 4: Podospora anserina Model

The yeast Saccharomyces cerevisiae, used in examples 1 to 3 does not have complex I of the mitochondrial respiratory chain. EBS therefore was tested on the strict filamentous mold Podospora anserina.

The strain used in the following examples contains a specific mutation modeling a human mutation in the sub-unit NDUFV1 of complex I leading to a mitochondrial illness. This strain has a growth defect at a non-permissive temperature greater than 31.5° C.

P. anserina Mutated Strain:

nuo-51^{A357V}: The mutation A357V was introduced into the gene nuo-51 of the wild type strain S along with a resistance cassette of nourseothricin (NatR). In order to mimic the human illness NDUFV1A341V, the genes NDI-1 and AOX were deactivated (El-Khoury et al., (2008) Curr Genet. 53:249-58)

Genotype of the strain: S, mat⁻, nuo-51^{A357V}, Δndi-1, Δaox, Nat^{R}, Hygro^{R}

### Materials and Methods

The heat sensitive mutant Pa nuo-51^{A357V} was homogeneously spread on sterile glass beads on a square Petri dish containing a solid minimal medium (M2). Small sterile filters were then placed on the agar-agar surface and 20 nmoles of EBS were added. DMSO (the vehicle of the compound) was used as a negative control. The plate was then incubated at the non-permissive temperature of 33° C. for 4-5 days then photographed.

The mutant Pa nuo-51^{A357V} was then cultivated at 32° C. where the growth thereof was reduced with respect to the wild type (WT) strain. The effect of EBS on the speed of growth of the mutant was determined on a supplemented minimal medium or not with increasing concentrations of EBS and estimated in centimeters of growth per day (cm/day).

### Results

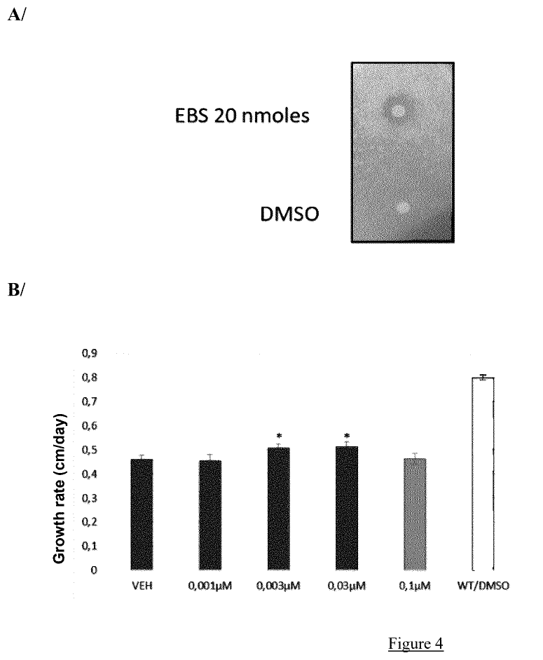

FIG. 4A shows the positive effect of EBS on the growth of the mutant. More precisely, between 0.003 and 0.03 μM, EBS demonstrated a significant improvement in the growth of the mutant Pa nuo-51^{A357V} (FIG. 4B).

### Example 5: Human Cell Models

### Materials and Methods

Statistical analyses were performed using the statistical test ANOVA with two mixed factors, applying the Bonferroni multiple test correction or with the graphpad software prism 9.5.1.

The number of stars indicates the value of the p-value <0.05 (*), <0.001 (**), <0.0001 (***), <0.0001 (****).

### Fibroblasts and Myoblasts of TAZ Patients:

EBS was tested for the effect thereof on the respiratory growth of fibroblasts of TAZ patients, as opposed to fibroblasts from eight different patients, carriers of mutations in the TAZ gene (suffering from Barth's syndrome). The data show the percentage of growth compared to T0.

The rate of proliferation was measured in a medium with a weak concentration of glucose (1 g/L; to force cells to use the OXPHOS system rather than glycolyse) in 384 well micro-titration plates (1200 cells/wells at T0) using the Sulforhodamine B (SRB) staining protocol at 24, 48, 72 or 96 hours after placing the cells with or without 12.15 nM of EBS.

The rate of proliferation was then measured under the same experimental conditions but with variable concentrations of EBS: The cells were analyzed 96 hours after treatment with increasing doses of EBS (from 150 pM to 234.9 μM). The data are the average of 8 patients or 3 controls in triplicate, in 4 independent experiments.

The rate of proliferation was then measured under the same experimental conditions but with variable concentrations of ethaselen: The cells were analyzed 96 hours after treatment with increasing doses of ethaselen (from 150 pM to 26.1 μM). The bars represent the average of 4 replicates coming from 2 patients in 3 independent experiments.

The levels of mitochondrial ATP in the fibroblasts of TAZ patients were evaluated using a Promega CellTiter-Glo kit. The cells in 96 well plates (5000/well) were treated or not treated with 12.15 nM of EBS for 96 hours then were incubated in the presence of Antimycin A (100 μM), of Rotenone (0.5 μM) or oligomycin A (3 μM).

EBS was also tested for the ability thereof to restore cell growth of a line of myoblasts from patients, carriers of mutations in the TAZ gene. The rate of proliferation was measured in a medium with a weak concentration of glucose in 384 well micro-titration plates (1200 cells/wells at T0) using the Sulforhodamine B (SRB) staining protocol at 48, 72, 96 or 120 hours after placing the cells. The graph shows the growth rate at 120 hours corresponding to the average of 3 independent experiments.

### KO (“Knock-Out”) Cell Line FMC1:

The cell line HeLa KO FMC1, mimicking human illnesses related to a deficiency of ATP synthase (NARP, MILS syndromes), were generated by the CRISPR-Cas9 method. The cell line HeLa KO FMC1 was cultivated in a DMEM medium containing 4.5 g/L of glucose and 110 mg/l of pyruvate and 50 mg/l of uridine.

The rate of proliferation of this HeLa cell line in which the gene FMC1 was deleted was measured in a medium containing either a low concentration of glucose (1 g/L), or of galactose in micro-plates of 384 wells (4500 cells/cm² at T0) using the Sulforhodamine B (SRB) staining protocol 96 hours after seeding and treatment of the cells. The bars represent the average of 8 replicates coming from 3 independent experiments. The data show the percentage of growth compared to T0.

### KO (“Knock-Out”) Cell Line CI (Complex I):

EBS was also tested for the ability thereof to restore mitochondrial respiration of fibroblast lines of patients carrying mutations of the nuclear gene NDUFV1 of mitochondrial complex I. The human lines NDUFV1 mimicking the human illnesses related to a mitochondrial deficit of complex I (Leigh's syndrome) are carriers of genetic mutations affecting the gene NDUFV1. These cell lines were cultivated in a DMEM medium containing 1.0 g/L of glucose and 1 mM of sodium pyruvate and 50 μg/ml of uridine (Sigma Aldrich, Lyon, France).

The respiration was measured using a Seahorse XF96 extracellular flow metabolic analyzer in 96 well plates according to the manufacturer's protocol (Agilent Technologies). Deficient cells were exposed for 48 h to different concentrations with a dose response curve associating a range of concentrations from 25 nM to 10 μM. (A) shows the rate of oxygen consumption, (B) the basal respiration, (C) respiration related to ATP synthase and (D) maximum respiration. The bars represent the average of 4 independent replicates.

Primary fibroblasts in exponential growth phase were detached by adding 2 ml of trypsin at 0.05%. Said fibroblasts were used to seed the XF96 plates (30×10³ cells/well) kept in culture for 4 hours. The cells were then incubated in DMEM without bicarbonate (Sigma-Aldrich) supplemented with 5.5 mM of glucose, 2 mM of L-glutamine, in an incubator without CO2 for 1 h.

The measurement of the oxygen consumption rate (OCR) was recorded under basal conditions to evaluate the mitochondrial respiratory activity and then the cells were treated sequentially with 4 μg/mL of oligomycin and 1.5 μM of carbonyl cyanide p-(trifluoromethoxy) phenylhydrazone (FCCP) (Sigma-Aldrich). The basal OCR, the respiration related to ATP, the maximum respiratory capacity and the respiration by proton leak were determined. Non-mitochondrial respiration (OCR after treatment with 2 μg/ml of Antimycin A) was subtracted from all OCR measurements. Respiration related to ATP (R-O) production was estimated from the difference between the basal respiration rates and inhibited by oligomycin and the respiration by proton leak was obtained by subtracting the non-mitochondrial respiration from the ROC measured after treatment with oligomycin. Maximum respiratory capacity has been determined as the rate of respiration in the presence of uncoupling FCCP. The results have been normalized depending on the number of cells.

### Results

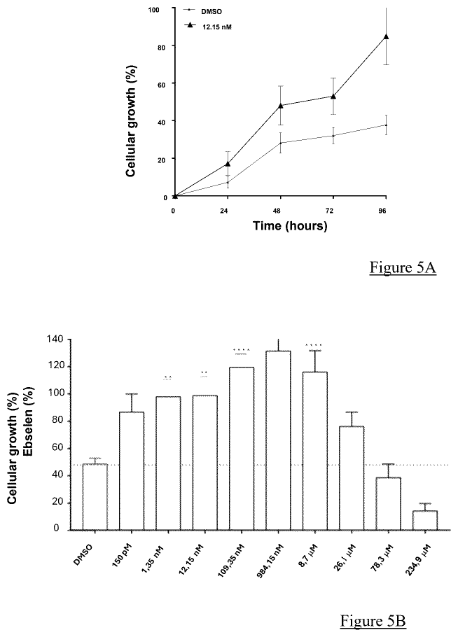

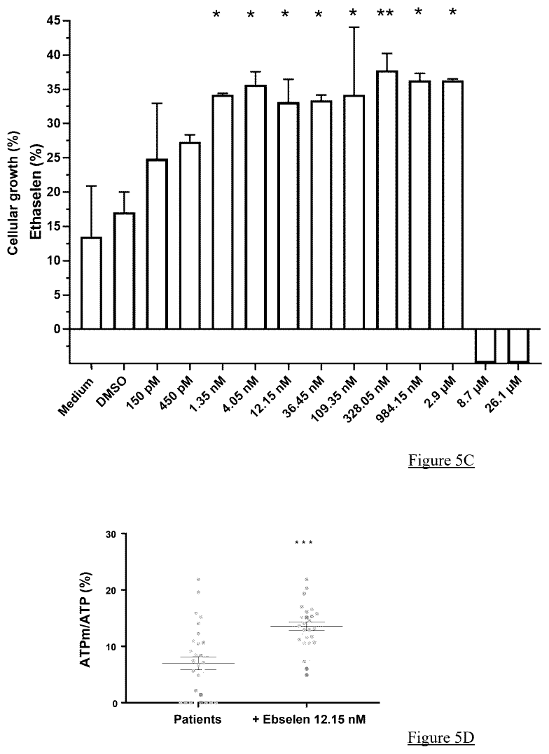

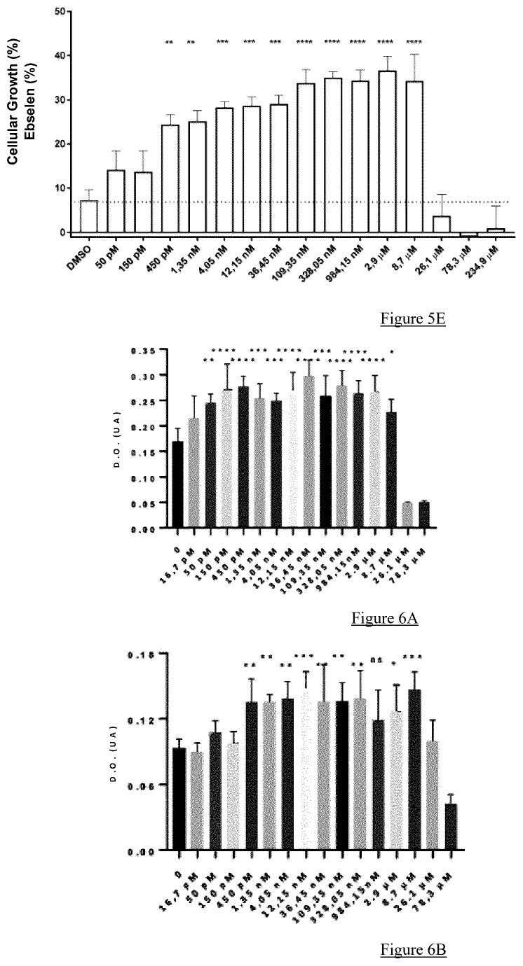

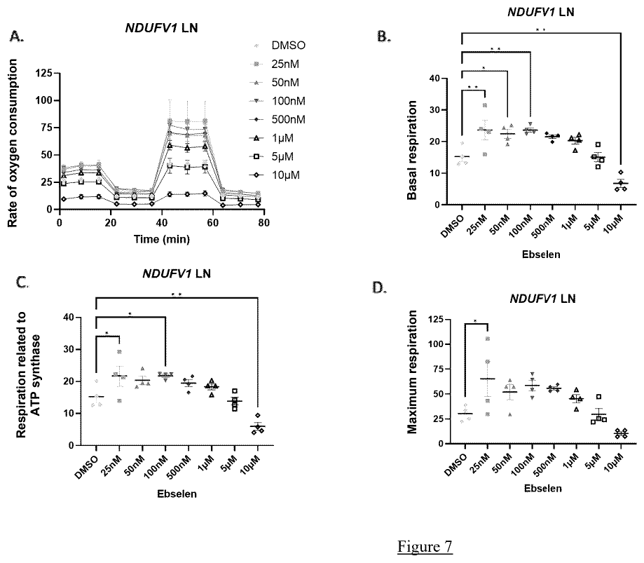

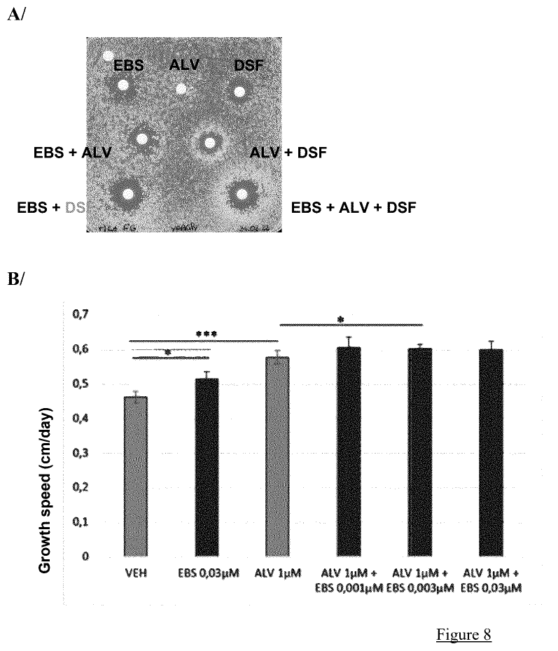

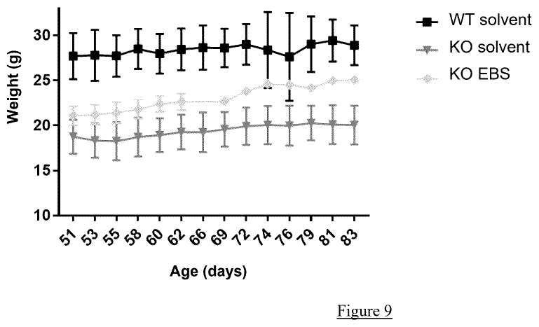

FIG. 5A shows that EBS at 12.15 nM improves the respiratory growth of fibroblasts of TAZ patients. At this concentration, EBS is effective after 24 hours and the improvement of cell proliferation is more pronounced at three other sampling times (48, 72 and 96 hours).

FIG. 5B shows that at 96 hours, EBS improves the respiratory growth of fibroblasts of TAZ patients over a wide range of concentrations: EBS has beneficial effects from 1.35 nM to 8.7 μM with toxicity beyond 234.9 μM, with respect to the negative control DMSO.

FIG. 5C shows that at 96 hours, ethasalen improves the proliferation of cells of patients suffering from Barth's syndrome over a wide range of concentrations going from about 1 nM to 3 μM.

FIG. 5D shows that EBS at concentration 12.15 nM improves the mitochondrial ATP production in fibroblasts of TAZ patients.

The effect of EBS on the restoration of the growth of a line of myoblasts of patients carrying mutations in the TAZ gene is illustrated in FIG. 5E: it shows that EBS from 450 μM to 8.7 μM has beneficial effects compared with the negative control DMSO, with a toxicity beyond 78.3 μM.

FIG. 6 shows that ebselen improves the proliferation of model cells of an ATP synthase deficiency (NARP, MILS syndromes) and in a dose-dependent manner and over a wide range of concentrations (pM to μM).

FIG. 7 shows that ebselen improves the respiration of model cells of mitochondrial pathologies related to complex 1, from 25 nM to 100 nM and becoming toxic beyond 10 μM.

### Example 6: Synergies Between Different Compounds

Disulfirame (DSF) and alverine (ALV) have been reported in the documents WO2020/254632 and WO2022/018297, respectively, as useful in treating pathologies associated with a mitochondrial malfunction.

Consequently, a possible synergistic effect between ebselen (EBS) and said compounds was tested in different models:
in a taz mutant yeast model
in a mutant of complex I of Podospora anserina (Pa nuo-51^{A357V})
in a taz mutant mammalian model.

### Materials and Methods

### Taz Yeast:

As in FIG. 1, the taz1 mutant yeast strain was spread over a respiratory solid medium containing ethanol as a carbon source. The strain was then exposed to filters on which were deposited either disulfirame (DSF 5 nmoles), ebselen (EBS 30 nmoles) or alverine (ALV 30 nmoles), alone or in association by two or three. The dishes were then incubated at the temperature of 36° C. then scanned after 5 days of incubation. DMSO (solvent) was used as negative control (upper left-hand filter).

Pa nuo-51^{A357V}:

The growth rates at 32° C. estimated in centimeters per day, were determined in a minimum medium supplemented with EBS (0.003 μM, 0.01 μM or 0.03 μM), of ALV (1 μM) or a combination of concentrations of both, compared with the same concentration of DMSO (VEH) per dish.

### Taz Mammalian Cell:

EBS, ALV and DSF were tested for their synergistic effect on respiratory growth of myoblasts from TAZ patients carriers of mutations in the TAZ gene. The rate of proliferation was measured in a medium with a weak concentration of glucose (to force cells to use the OXPHOS system rather than glycolyse) in 384 well micro-titration plates (1200 cells/wells at T0) using the Sulforhodamine B (SRB) staining protocol at 120 hours after seeding the cells with or without lone compounds or compounds in association at the concentrations indicated.

### Results

FIG. 8A shows that the combination of the 3 molecules EBS, ALV and DSF increased the growth halo around the filter where same were deposited in association, suggesting a synergistic effect of the 3 candidate drugs on the respiratory growth of the tafazzin deficient yeast model.

FIG. 8B shows that the combination of 1 μM ALV+0.003 μM EBS significantly increased the growth of the mutant Pa nuo-51^{A357V}, compared to 1 μM ALV alone suggesting a synergistic effect on the growth of said complex I mutant.

### Example 7: Mouse Model of Barth Syndrome

### Materials and Methods

Mice lacking the TAZ gene (KO model of Barth syndrome) or wild type (WT) gene were force-fed twice a day with 10 mg/kg of Ebselen as of an age of 51 days. The weight was regularly monitored during 1 month of treatment.

### Results

As shown in FIG. 9, the KO mice with EBS seemed to take on more weight than mice treated with the solvent.

1. A pharmaceutical composition comprising a compound having a moiety of formula: 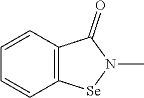
for use in the treatment of an illness associated with mitochondrial malfunction.

2. The composition for the use thereof according to claim 1, according to which the compound is of formula: 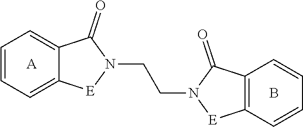
in which
E est O, S, SO, SO₂, Se or SeO
each of the phenyl rings A and B being possibly substituted by one or a plurality of substituent,
in which each substituent is chosen independently from amongst:
a halogen, which is preferably chosen from amongst F, Cl and Br
an alcohol
an amine
a nitro
a C1-C4 alkyl such as a C1-C2 alkyl or a C1 alkyl, possibly substituted by one or a plurality of halogen atoms, of which each is preferably chosen from amongst F, Cl and Br; and
a C1-C4 alkoxy, such as a C1-C2 alkoxy or a C1 alkoxy, possibly substituted by one or a plurality of halogen atoms, of which each is preferably chosen from amongst F, Cl and Br.

3. The composition for the use thereof according to claim 2, according to which the compound is ethaselen or one of the derivatives thereof, e.g. MAD423.

4. The composition for the use thereof according to claim 1, according to which the compound is of formula: 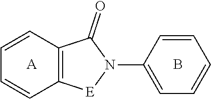
in which
E est O, S, SO, SO₂, Se or SeO
each of the phenyl rings A and B being possibly substituted by one or a plurality of substituent,
in which each substituent is chosen independently from amongst:
a halogen, which is preferably chosen from amongst F, Cl and Br
an alcohol
an amine
a nitro
a C1-C4 alkyl such as a C1-C2 alkyl or a C1 alkyl, possibly substituted by one or a plurality of halogen atoms, of which each is preferably chosen from amongst F, Cl and Br; and
a C1-C4 alkoxy, such as a C1-C2 alkoxy or a C1 alkoxy, possibly substituted by one or a plurality of halogen atoms, of which each is preferably chosen from amongst F, Cl and Br.

5. The composition for the use thereof according to claim 4, according to which the compound is:
2-phenyl-1, 2-benzisoselenazol-3(2H)-one or ebselen of formula: 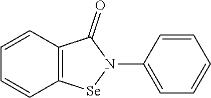 [SMILES: O=c1c2ccccc2[se]n1-c1ccccc1]
or
1-oxide-2-phenyl-1, 2-benzisoselenazol-3(2H)-one or ebselen or ebselen selenoxide of formula:

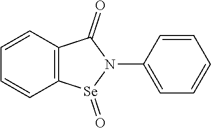 [SMILES: O=C1c2ccccc2[Se](=O)N1c1ccccc1]

6. The composition for the use thereof according to claim 1, according to which the illness is an illness of the mitochondrial respiratory chain, advantageously associated with a complex I deficiency.

7. The composition for the use thereof according to claim 1, according to which the illness is a genetic illness.

8. The composition for the use thereof according to claim 7 according to which the genetic illness comprises at least one mutation in at least one of the following genes: MTND1(or ND1), MTND2(or ND2), MTND3(or ND3), MTND4(or ND4), MTND5(or ND5), MTND6(or ND6), MTND4L(or ND4L), NDUFA1, NDUFA2, NDUFA3, NDUFA4, NDUFA5, NDUFA6, NDUFA7, NDUFA8, NDUFA9, NDUFA10, NDUFA11, NDUFA12, NDUFA13, NDUFAB1, NDUFB1, NDUFB2, NDUFB3, NDUFB4, NDUFB5, NDUFB6, NDUFB7, NDUFB8, NDUFB9, NDUFB10, NDUFB11, NDUFC1, NDUFC2, NDUFS1, NDUFS2, NDUFS3, NDUFS4, NDUFS5, NDUFS6, NDUFS7, NDUFS8, NDUFV1, NDUFV2, NDUFV3, NDUFAF1, NDUFAF2, NDUFAF3, NDUFAF4, NDUFAF5, NDUFAF6, NDUFAF7, NDUFAF8, NUBPL, ACAD9, TMEM70, TMEM126B, FOXRED1, ECSIT, AIF, TIMMDC1.

9. The composition for the use thereof according to claim 7 according to which the genetic illness comprises at least one mutation in at least one of the following genes: MTTL1, APT6, FMC1, TAZ, COX2, SURF1, POLG, MPV17, OPA1, COA6, ND6 and BCS1L, advantageously ATP6, FMC 1, TAZ, COX2, SURF1 OR MPV17.

10. The composition for the use thereof according to claim 1 according to which the illness is chosen from amongst the group composed of: MELAS syndrome, myopathy or cardiomyopathy inherited from the mother, NARP or MILS syndrome, Leigh's syndrome, Leber hereditary optic neuropathy (LHON), Barth's syndrome, the depletion of mitochondrial DNA syndrome in particular 4A (Alpers type) and 4B (MNGIE type), mitochondrial recessive ataxia syndrome, sensory ataxic neuropathy, dysarthria and ophthalmoplegia, spinocerebellar ataxia with epilepsy, progressive external ophthalmoplegia, mitochondrial DNA-6 depletion syndrome, Navajo type neuropathy, Behr's syndrome, mitochondrial DNA 14 depletion syndrome, infantile cardioencephalomyopathy due to a deficit of cytochrome c oxydase (COA6 mutations), mitochondrial type 1 complex III nuclear deficiency, GRACILE syndrome and Bjornstad's syndrome.

11. The composition for the use thereof according to claim 10, according to which the illness is chosen from the group constituted by: Leigh's syndrome, Leber Hereditary Optic Neuropathy (LHON), MELAS syndrome, NARO or MILS syndrome, Barth's syndrome, mitochondrial DNA depletion syndrome and Navajo type neuropathy.

12. The composition for the use thereof according to claim 1 according to which the illness is chosen from the group constituted by:
neurodegenerative illnesses such as Alzheimer, Parkinson's disease, Huntington's disease, amyotrophic lateral sclerosis (Lou Gherig's disease) and Friedreich's ataxia;
cardiovascular illnesses, such as atherosclerosis and other cardiac and vascular ailments;
diabetes and metabolic syndrome;
auto-immune illnesses, such as multiple sclerosis, systemic lupus erythematosus and type 1 diabetes;
neurobehavioral and psychiatric illnesses, such as autistic spectrum disorders, schizophrenia, bipolar and mood disorders;
gastro-intestinal disorders;
fatigue illnesses such as chronic fatigue syndrome and Gulf War illnesses;
muscular-skeletal illnesses such as fibromyalgia and hypertrophy/atrophy of the skeletal muscles;
muscular dystrophies,
cancer; and
chronic infections.

13. The composition for the use thereof according to claim 1, according to which the composition comprises another compound for treating the same illness, advantageously alverine and/or disulfirame.

14. The composition for the use thereof according to claim 1, according to which the composition is administered orally.

15. The composition for the use thereof according to claim 1, according to which the composition is in a solid form such as a tablet, again more advantageously comprising 200 mg of ebselen.
